(19) United States
(12) Patent Application Publication
(10) Pub. No.: US 2004/0144392 A1
(43) Pub. Date: Jul. 29, 2004

(54) Breast nipple duct protective device

(76) Inventor: Richard L. Mueller JR., Jackson, WY (US)

Correspondence Address: OLSON & HIERL, LTD., 36th Floor, 20 North Wacker Drive, Chicago, IL, 60606, US

(21) Appl. No.: 10/349,859

(22) Filed: Jan. 23, 2003

(51) Int. Cl. A61F 5/56

(52) U.S. Cl. 128/848

(57) ABSTRACT

A breast nipple duct protective device of the present invention comprises a post having a longitudinal axis, a distal end and a proximal end. The post has an anchor at the distal end, and a grip at the proximal end. The post has an outside diameter of no more than about 0.5 millimeters, and the anchor has a transverse dimension at least about 1.25 times the outside diameter of the post. The transverse dimension of the anchor is selected to be small enough to be pushed through the nipple duct orifice without causing damage to the duct, but large enough to hold the device in place in the duct for a prolonged period of days to weeks. The grip is dimensioned to prevent the device from entering the duct any further than the sum of the lengths of the post and the anchor. The anchor can have an articulating form with an adjustable transverse dimension. The device may also be utilized as a decorative breast nipple ornament.

### FIELD OF THE INVENTION

This invention relates to devices for protecting and marking breast nipple ducts. More particularly, this invention relates to devices suitable for placement within a breast nipple duct.

### BACKGROUND

Breast cancer is one of the health threats most feared by American women, and is the most common form of cancer in women. A key to treatment is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. While breast cancer is most common among women, in rare instances the human male may also have occurrences of breast cancer.

Other methods of detecting breast cancer are based on the fact that a vast majority of instances of breast cancer begins in the lining of mammary ducts. Studies have shown that an estimated 80% to 90% of all breast cancers occur within the intraductal epithelium of the mammary glands and that in such cases fluid within the mammary duct contains high levels of breast cancer markers. Fluid within the breast ducts contains an assemblage and concentration of hormones, growth factors and other potential markers comparable to those secreted by, or acting upon, the surrounding cells of the alveolar-ductal system. Likewise, mammary duct fluid typically contains cells and solid cellular debris, or products that can also be used in cytological or immunological assays.

A typical breast nipple contains about 8 to about 12 orifices that are external termini of the mammary ducts through which milk and other mammary fluid flow during lactation, for example. The orifices are each surrounded by sphincter muscle which normally keeps the ducts closed. Application of pressure posterior to the periphery of the sphincter muscles can cause the muscles to open. Samples of mammary fluid and/or mammary duct cells can be collected for analysis by insertion of a catheter into a mammary duct lumen through the breast nipple duct orifice. A catheter of a diameter sufficiently small enough to be inserted into a breast nipple duct (i.e., typically having a diameter of no more than about 1 millimeter) is positioned within the lumen of a mammary duct and a fluid sample or cells can be collected in the catheter. This procedure is commonly referred to as ductoscopy.

In many instances, a clinician may desire to take samples from the same duct over a period of time. This requires that the clinician be able to find the same duct repeatedly, and that the duct must be reopened each time as well. When a catheter is inserted into a breast nipple duct, the tissue around the orifice can be abraded, however, and can heal over (i.e., close up), making it difficult to insert a catheter therethough at a future date. In addition, the ducts typically are not very visually distinct nor uniformly distributed, thus, a clinician must typically make an accurate diagram of the nipple in order to relocate the duct at a future date. There is, therefore, a great, unfulfilled need for a safe, convenient, and reliable device for marking a breast nipple duct and for protecting the duct orifice from closing after ductoscopy so that the duct can be relocated at a future date for additional clinical testing. The present invention fulfills this need.

### SUMMARY OF THE INVENTION

A breast nipple duct protective device of the present invention comprises a post having a longitudinal axis, a distal end and a proximal end. The post has an anchor at the distal end thereof, and a grip at the proximal end. The post has an outside diameter of no more than about 0.5 millimeters, and the anchor has a transverse dimension at least about 1.25 times the outside diameter of the post, preferably in the range of about 0.3 to about 1 millimeter (mm). The transverse dimension of the anchor is selected so as to pass through the nipple duct orifice and the sphincter muscle without causing damage to the duct, but large enough to hold the device in place in the duct for a prolonged period of time, e.g. days to weeks. The grip is dimensioned to serve as a stop which prevents the device from entering the duct any further than the sum of the lengths of the post and the anchor. The device is useful in a clinical setting for marking a breast nipple duct after a mammary duct cell or fluid sample has been collected, so that a clinician can relocate the same breast nipple duct at a future date, e.g., to take another cell or fluid sample from the same duct. In addition, the presence of the device within the duct prevents the orifice from closing after ductoscopy.

In one preferred embodiment, the breast nipple protective device comprises an elongated rod having rounded or blunt termini at both ends thereof, the rod having an outside diameter of no more than about 0.5 millimeters and the termini having an outside diameter at least about 1.25 times the outside diameter of the rod. At least one terminus is dimensioned to be inserted through the nipple duct orifice with a relatively small amount of force being applied to the sphincter muscles of the duct. Preferably, the other terminus is larger than the terminus that is dimensioned to be inserted through the duct orifice, and is dimensioned such that, as a practical matter, it will not naturally fit through the duct orifice. This relatively larger terminus acts as a stop, preventing the device from being inserted beyond the total length of the smaller terminus and the rod. The larger terminus also acts as a visual marker for the nipple duct. The relatively smaller terminus keeps the device secured within the duct so that it will remain in place for a prolonged period of time.

In another preferred embodiment, the breast nipple duct protective device of the present invention includes a hollow post having a longitudinal axis, a distal end, a proximal end and defining a lumen. An articulating anchor having an adjustable transverse dimension is connected to the distal end of the post and a grip is attached to the proximal end of the post. The grip included an actuator adapted for adjusting the transverse dimension of the articulating anchor. The actuator is operably connected to the articulating anchor through the lumen of the post Preferably the post has an outside diameter of no more than about 0.5 millimeters; the articulating anchor has a minimum transverse dimension that is less than 1.25 times the outside diameter of the post and a maximum transverse dimension of at least about 1.25 times the outside diameter of the post.

In use, a clinician can locate a breast nipple duct, catheterize the duct, and recover a mammary duct cell and/or mammary fluid sample therefrom. The clinician can then remove the catheter and then can mark the duct by inserting therein a device of the present invention. The clinician grasps the larger terminus or grip manually or with a implement (e.g., forceps, a pin vise, and the like), as the case may be, and inserts the relatively smaller terminus or anchor through the sphincter and into the lumen of the duct. The depth to which the device is inserted in the duct is controlled by the length of the post or rod. The larger terminus or grip acts as a stop, which prevents the device from being inserted too far (i.e., beyond the external opening of the duct). The device then remains in place in the duct until the clinician removes it (i.e., when the next cell or fluid sample is to be taken, or when it is determined that no further samples need to be taken).

In an alternative use, the device of the present invention can serve as a form of body art or jewelry, i.e. a decorative breast nipple ornament. The grip can have a decorative form, such as a setting for a gem stone or semiprecious stone, or can have any other aesthetically pleasing form, as desired by the wearer (i.e., breast nipple jewelry).

### BRIEF DESCRIPTION OF THE DRAWINGS

In the Drawings, FIG. 1 is a cross-sectional view of a human breast having breast nipple protective devices of the invention disposed within mammary ducts therein;

FIGS. 2-6 are side views of preferred embodiments of the breast nipple protection devices of the present invention, having different configurations of the anchor and grip members;

FIGS. 7 and 8 are partial cut-away views of a breast nipple protection device of the present invention having an articulating anchor; FIG. 7 shows the anchor in closed position and FIG. 8 shows the anchor in open position;

FIG. 9 is a side view of another preferred embodiment of the breast nipple protection devices of the present invention, including a passageway for a guide wire through the longitudinal axis of the device;

FIGS. 10 and 11 are cross-sectional views of another embodiment of a breast nipple protection device of the present invention having an articulating anchor in the form of an adjustable diameter loop; FIG. 10 shows the device in resting position and FIG. 11 shows the device in working position;

FIGS. 12 and 13 are side views of a device of the present invention having a grip including a removable retaining clip, and an articulating anchor in the form of resilient bands longitudinally arranged at the distal end of the post an connected to an actuator in the grip; FIG. 12 shows the device in resting position with the retaining clip disengaged from the grip; FIG. 13 shows the device in working position, with the retaining clip engaged with the grip;

FIGS. 14 and 15 are side views of a device of the present invention having a grip including a cradle, and an articulating anchor in the form of resilient bands helically arranged at the distal end of the post and connected to an actuator in the grip; FIG. 14 shows the device in resting position with the actuator disengaged from the cradle; FIG. 15 shows the device in working position with the actuator engaged with the cradle;

FIGS. 16 is a side view of a preferred embodiment of the device of the present invention having a hollow post connected to a grip having a transverse aperture and a through-hole, both in open communication with the hollow post, and an anchor in the form of a bead on a string; the device is depicted with the bead held against the open distal end of the post by the string in resting position;

FIG. 17 shows the device of FIG. 16 in working position within a mammary duct; the threaded bead anchor is displaced from the longitudinal axis of the post, and abutting the post at the sphincter of the duct.

### DETAILED DESCRIPTION OF PREFERRED EMBODIMENTS

A breast nipple duct protective device of the present invention comprises a post having a longitudinal axis, a distal end and a proximal end. The post has an anchor at the distal end, and a grip at the proximal end. The post preferably has an outside diameter of no more than about 0.5 mm, and the anchor has a transverse dimension at least about 1.25 times the outer diameter of the post. In one preferred embodiment the anchor has a transverse dimension of about 1.4 to about 2 times larger than the outside diameter of the post. The transverse dimension of the anchor is small enough to pass through the nipple duct orifice without injuring the duct, but large enough to retain the device in place for an extended time period. The grip is dimensioned to prevent the device from entering the duct any further than the sum of the lengths of the post and the anchor.

The anchor can be configured in any form that can be safely inserted through the orifice of a breast nipple duct and into a mammary duct lumen. Preferably the anchor has a generally blunt, atraumatic end that does not traumatize the tissue of the nipple and the mammary duct. Due to the size limitations of human breast nipple ducts, the transverse dimension of the anchor is selected to be insertable through the nipple duct orifice without such force as might damage the duct tissue. The human mammary duct generally has a sinus region having a slightly larger inside diameter anterior to and spaced from the duct orifice and sphincter muscles of the duct. The transverse dimension of the anchor is selected to fit snugly within the relatively larger diameter region of the duct. Preferably, the transverse dimension of the anchor is at least 1.25 times the outer diameter of the post, more preferably in the range of about 0.3 to about 1 mm.

The anchor can have any shape that is convenient for insertion through the orifice of the nipple duct and holds the device in place within the duct. The anchor can have a generally bulbous shape, such as spheroid, ellipsoid, pyriform (e.g., pear-shaped), oviform, and the like. Alternatively, the anchor can have a generally arciform shape, such as a forked shape, a crescent (e.g., curved or hooked) shape, and the like. Alternatively, the anchor can have a virgate shape transversely offset from the longitudinal axis of the post (i.e., a bent rod shape). Preferably the extremity of the anchor is blunt or rounded to minimize the likelihood of trauma to the duct tissue upon insertion.

In some preferred embodiments, the anchor can have an expandable, articulating form wherein the transverse dimension of the anchor can be adjusted to facilitate insertion. For example, the transverse dimension of the anchor can be minimized to a closed or resting position during insertion through the duct orifice. When the device has been suitably disposed within the duct, the transverse dimension of the anchor then can be increased to an open position or working position. In this manner, the breast nipple protection device can be secured in position within the duct. When a clinician desires to remove the device, for example to perform another ductoscopic examination, the anchor can be articulated into its closed configuration to facilitate removal of the device from the duct.

The post is preferably a rod, wire, or tube, preferably having an outer diameter in the range of about 0.2 to about 0.5 mm. The post can be relatively rigid or flexible as desired. When the rod is flexible, it can be resilient, taking on a new configuration when a force is applied transversely thereto, but having a specified straight or curved configuration to which the post returns after the deforming force is relieved. Alternatively, the flexible post can be pliable, rather than resilient, such that the shape or configuration of the post can be changed by applying a force transverse to the longitudinal diameter of the post. In this case the post retains its new configuration when the force is relieved. Usually the post is no more than about 20 millimeters long. Preferably the post is about 5 to about 20 millimeters long, more preferably about 5 to about 10 millimeters long.

The grip can comprise any convenient configuration for grasping the device during insertion into a breast nipple duct and during removal of the device from a breast nipple duct. Preferably the grip has a transverse dimension at least about 1.25 times the outer diameter of the post. In particularly preferred embodiments, the grip has a transverse dimension that is greater than the transverse dimension of the anchor. The grip can be solid, apertured, or hollow. In one embodiment, the grip defines a recessed aperture that is in open communication with a passageway in the post and facilitates size adjustment of the anchor during insertion and eventual removal of the device.

The device can include an open passageway traversing its entire length, which can be used in conjunction with a guide wire. A clinician can place a guide wire in a mammary duct after ductoscopy to mark a site of ductal wall carcinoma for later return to that site. The nipple protective device then can be guided into the nipple duct by threading the guide wire through the passageway and sliding the device along the guide wire into the duct. Alternatively, such a passageway can be removably capped, providing a means for flushing the duct with an antiseptic fluid, a chemotherapeutic agent, and the like.

In another embodiment, the grip member defines a transverse through aperture that can be used to aid in removal of the device, for example. In a nipple jewelry embodiment of the present invention, the aperture can be oriented transverse to the longitudinal axis of the post and can contain a decorative ring, or other loose, relatively free-moving form for attachment of dangling decorative items in a form similar to conventional earing jewelry. The grip can also be in the form of a setting for a gem stone or a semiprecious stone, for example, particularly in a nipple jewelry embodiment of the present invention. The device of the present invention has an advantage over current forms of breast nipple jewelry, which often conventionally involve piercing of the nipple. The breast nipple jewelry of the present invention can be worn without piercing the nipple.

In another preferred embodiment, the breast nipple duct protective device of the present invention includes a hollow post having a longitudinal axis, a distal end, a proximal end and defining a lumen. An articulating anchor having an adjustable transverse dimension is connected to the distal end of the post and a grip is attached to the proximal end of the post. The grip includes an actuator adapted for adjusting the transverse dimension of the articulating anchor. The actuator is operably connected to the articulating anchor through the lumen of the post. Preferably the post has an outside diameter of no more than about 0.5 millimeters; the articulating anchor has a minimum transverse dimension that is less than 1.25 times the outside diameter of the post and a maximum transverse dimension of at least about 1.25 times the outside diameter of the post.

The articulating anchor can be in the form of jaws, hingedly attached to the distal end of the post, or in the form of a loop, an expanding basket or cage, and the like. Regardless of the form of the articulating anchor, its transverse dimension in the closed or resting position should be small enough for a clinician to insert the anchor through the orifice of a breast nipple duct and into the lumen of the mammary duct. After insertion, the anchor is fixed or locked in the open or working position, in which the transverse dimension of the anchor is large enough to hold the device securely in place in the duct for an extended period of time.

In yet another embodiment, the breast nipple protection device comprises an elongated rod having rounded or blunt termini at both ends thereof, the rod having an outside diameter of no more than about 0.5 millimeters and the termini having an outside diameter at least about 1.25 times the outside diameter of the rod. At least one terminus is dimensioned to be inserted through the nipple duct orifice with a relatively small amount of force being applied to the sphincter muscles of the duct. Preferably, the other terminus is larger than the terminus that is dimensioned to be inserted through duct orifice, and is dimensioned such that, as a practical matter it will not naturally fit through the orifice. This relatively larger terminus acts as a stop, preventing the device from being inserted beyond the total length of the smaller terminus and the rod. Additionally, the larger terminus acts as a visual marker for the nipple duct. The relatively smaller terminus keeps the device secured within the duct so that it will remain in place for a prolonged period of time. The smaller terminus can have an adjustable transverse dimension, and can be articulated to increase or decrease the transverse dimension as described above for the articulating anchor.

The devices of the present invention are useful in a clinical setting for marking a breast nipple duct after a mammary ductoscopy, so that a physician can relocate the same breast nipple duct at a future date, e.g., to take another cell or fluid sample from the same duct. In addition, the devices of the present invention can prevent the nipple duct from healing over after the ductoscopic procedure is complete. After ductoscopy, the clinician can protect and mark the nipple duct from which a fluid or cell sample has been collected by gently inserting the anchor end of device of the present invention through the duct orifice and sliding the device into the duct until the grip comes into contact with the nipple, at which point the device is suitably positioned to remain in place in the duct for a prolonged period of time, which can be days, weeks, or months in duration, if desired. The duct is marked for future location and is protected from healing over by the presence of the device within the duct. When the clinician desires to perform another ductoscopic examination in the same duct, the clinician can gently remove device and access the duct. Preferably the device is sterilized before insertion into the breast nipple.

The portions of the device of the present invention which are to be in direct contact with the nipple duct tissues are preferably constructed from a physiologically tolerable metallic or polymeric material. As used herein, and in the appended claims, the term “physiologically tolerable” in reference to materials of construction for breast nipple protective devices or decorative nipple jewelry means that the material will not elicit undesirable physiological responses such as swelling, rashes, or other allergic physiological responses. Preferably, the device of the present invention is made from a non-allergenic metal such as gold, stainless steel, platinum, or titanium, or the device can be gold-plated. Alternatively, the device can be constructed of a physiologically tolerable polymeric (e.g., plastic) material such as polyethylene, polypropylene, a fluorocarbon polymer, and the like. For clinical applications where magnetic resonance imaging (MRI) compatibility is desirable, a non-ferrous metal such as titanium, or a polymeric material with a strengthening titanium ribbon can be used.

Turning now to the drawings, FIG. 1 is a partial cross-sectional view of a human breast 2 in the region of nipple 3 and illustrating two breast nipple protective devices 10 and 20 disposed within mammary ducts 4 and 6, respectively. Mammary duct 4 has a sinus region 5 and mammary duct 6 has a sinus region 7. Anchor 14 of protective device 10 is situated within sinus region 5 abutting sphincter 8, and anchor 24 of protective device 20 is similarly situated within sinus region 7 abutting sphincter 9.

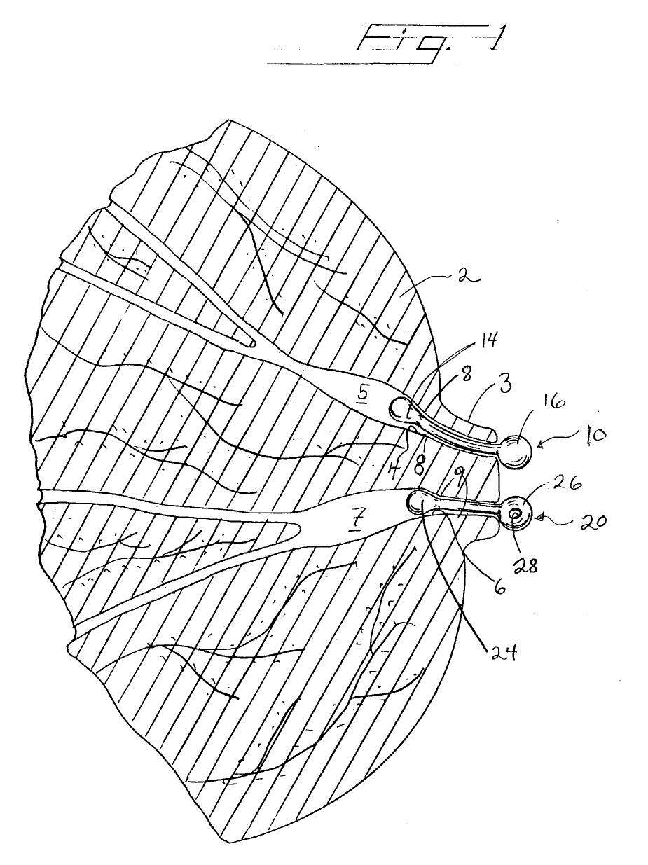

FIG. 2 is a side view of a preferred embodiment of the breast nipple protective device of the present invention. Device 10 includes a post or rod 12 having an anchor 14 at its distal end and a grip 16 at its proximal end. Anchor 14 has a generally bulbous, pyriform shape, having its narrower transverse dimension attached to or merging into the distal end of post 12. Grip 16 is generally spheroid in shape.

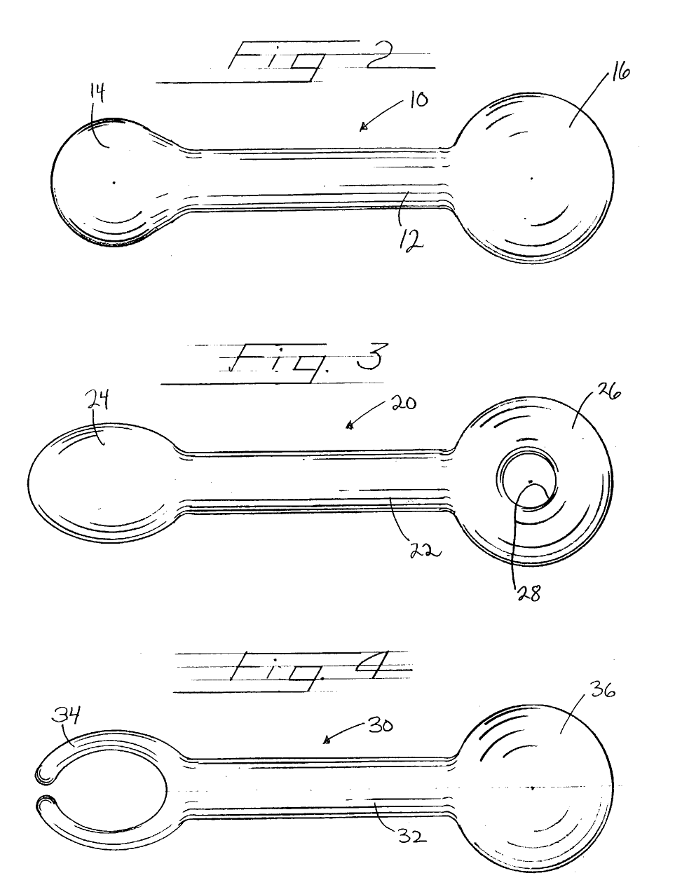

FIG. 3 is a side view of another preferred embodiment of the breast nipple protective device of the present invention. Device 20 includes a post or rod 22 having an anchor 24 at its distal end and a grip 26 at its proximal end. Anchor 24 has a generally bulbous, ellipsoid shape and grip 26 is generally spheroid in shape. Grip 26 also defines a through-hole 28 transverse to the longitudinal axis of post 22.

FIG. 4 is a side view of an alternative preferred embodiment of the breast nipple protective device of the present invention. Device 30 includes a post or rod 32 having a forked, arciform anchor 34 at its distal end and a spheroid-shaped grip 36 at its proximal end.

FIG. 5 is a side view of another alternative preferred embodiment of the breast nipple protective device of the present invention. Device 40 includes a post or rod 42 having an anchor 44 at its distal end and a grip 46 at its proximal end. Anchor 44 has a generally arciform, crescent shape. Grip 46 is generally spheroid in shape.

FIG. 6 is a side view of a yet another preferred embodiment of the breast nipple protective device of the present invention. Device 50 comprises a post or rod 52 having an anchor 54 at its distal end and a grip 56 at its proximal end. Anchor 54 has a generally bulbous, pyriform shape, oriented with its wider transverse dimension merging into post 52. Grip 56 has a generally spheroidal shape.

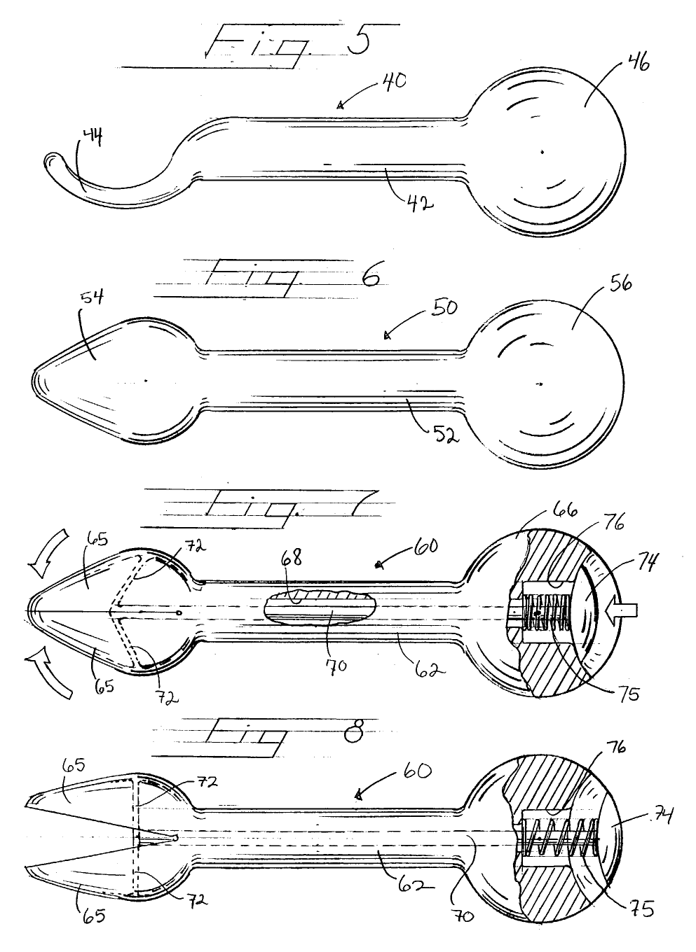

FIGS. 7 and 8 illustrate yet another preferred embodiment of the breast nipple protective device of the present invention having an articulating anchor, which allows the transverse dimension of the anchor to be adjusted. Device 60 is provided with a hollow post 62 having an anchor 64 at its distal end and grip 66 at its proximal end. Anchor 64 comprises two hemi-ellipsoidal jaws 65 which are hingedly attached to the distal end of post 62. Jaws 65 are hinged to post 62 and can be articulated into a closed position (shown in FIG. 7) or into an open position (shown in FIG. 8). Post 62 defines a lumen 68 in which a tie-rod 70 is slidably disposed. Tie-rod 70 is pivotally attached at its distal end to each of jaws 65 by cross-ties 72. At its proximal end, tie-rod 70 is attached to plunger 74 within recess 76 of grip 66 and through spring 75. Plunger 74 can be urged into a recessed position, in which tie-rod 70 is slid distally toward anchor 66 within lumen 68, closing jaws 65 and compressing spring 75, as shown in FIG. 7. Alternatively, plunger 74 can be released to a distended position, shown in FIG. 8, wherein tie-rod 70 is slid proximally away from anchor 66, pulling jaws 65 into an open position and spring 75 is in a relaxed position. Spring 75 maintains jaws 65 in the open position when plunger 74 is released.

In an alternatively embodiment, plunger 74 can be a screw and recess 76 can be threaded to complement the threads on the screw, and spring 75 can be omitted.

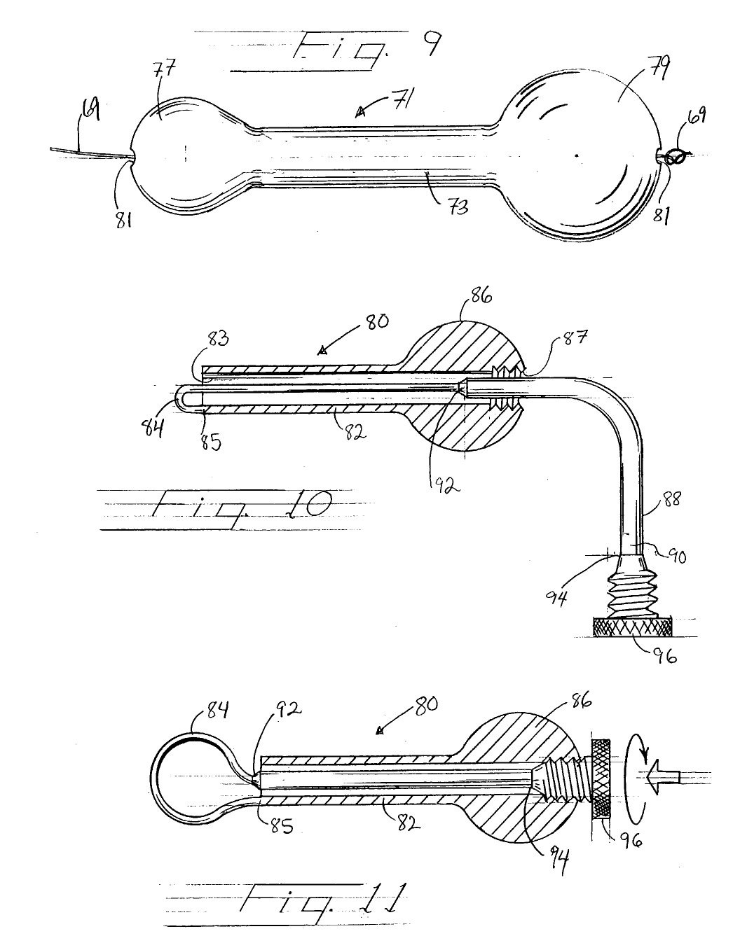

FIG. 9 is a side view of another preferred embodiment of the breast nipple protection device of the present invention. Device 71 includes a post 73 having bulbous anchor 77 at its distal end and spherical grip 79 at its proximal end. Anchor 77, post 73 and grip 79 collectively define a passageway 81 through the longitudinal axis of device 71. In use, guide wire 69 can be positioned within a mammary duct and device 71 can be guided into the duct by threading the guide wire 69 through passageway 81.

FIGS. 10 and 11 are cross-sectional views of another embodiment of a breast nipple protection device of the present invention. FIG. 10 shows device 80 in resting position. As shown in FIG. 10, device 80 includes an open, hollow post 82 defining a passageway 83. An articulating anchor 84, in the form of a flexible loop, is attached to the distal end 85 of post 82. Anchor 84 is shown substantially slidably disposed within passageway 83. Grip 86 is connected to the proximal end of post 82 and defines a threaded aperture 87 at the end passageway 83. Grip 86 is provided with an actuator 88, which includes flexible barrel 90 connected at its distal end 92 to the flexible anchor 84 and at its proximal end 94 to threaded cap 96. Cap 96 is threadably engagable with aperture 87. FIG. 11 shows device 80 in working position with cap 96 threaded into aperture 87 and anchor 84 deployed as a loop outside of passageway 83. Optionally, the distal end 92 of barrel 90 can be attached to anchor 84 by a pivoting joint such as a ball joint. Alternatively, the proximal end 94 of barrel 90 can be attached to cap 96 by a pivoting joint.

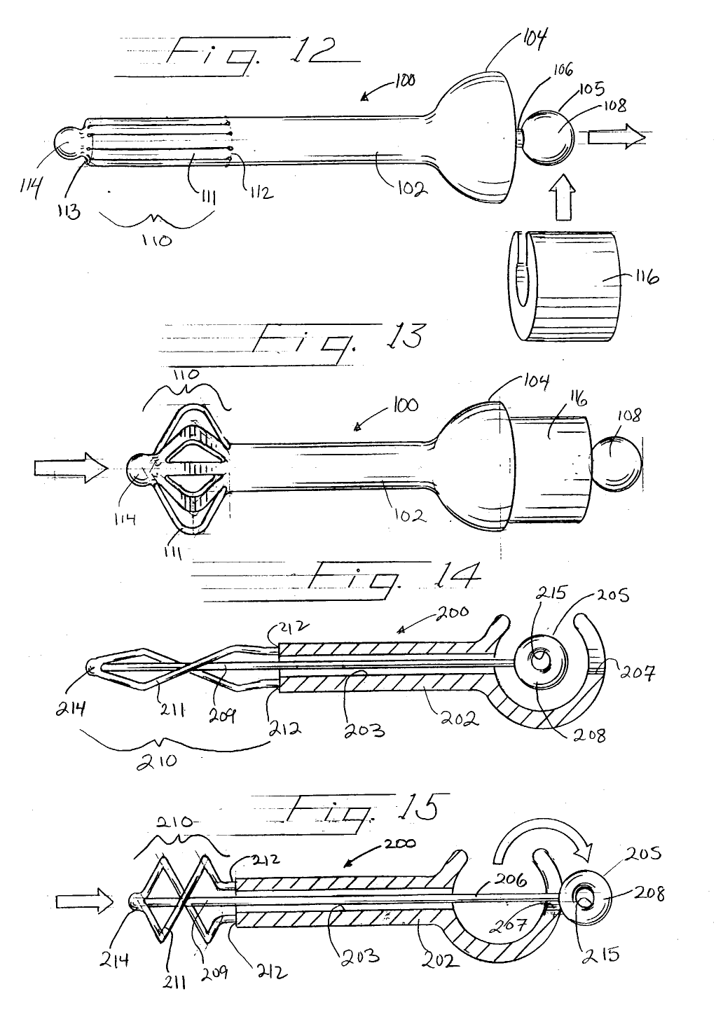

FIGS. 12 and 13 are side views of a device of the present invention having an alternative form of articulating anchor. Device 100 (shown in resting position in FIG. 12) includes a hollow post 102 having a hollow grip 104 at its proximal end. Grip 104 is provided with an actuator 105 including a tie rod 106 extending through the hollow grip 104 and hollow post 102. Tie rod 106 terminates in knob 108 at its proximal end. Articulating anchor 110, including a plurality of flexible bands 111 is situated at the distal end of post 102. The proximal end of each band 111 is connected to post 102, and the distal end 113 of each band is attached to the blunt, atraumatic distal end 114 of tie rod 106. Grip 104 also includes a removable retaining clip 116, which is adapted to hold actuator 105 in place when actuator 105 is passed proximally through the hollow post 102, as indicated by the arrows.

FIG. 13 shows device 100 in working position, with actuator 105 displaced proximally, as indicated by the arrow, and retaining clip 116 positioned over tie rod 106 and held in place by knob 108 and the resistive force provided by flexed bars 111 of anchor 110. In working position, anchor 110 has the form of an expandable cage.

In use, a clinician can insert device 100, in resting position (FIG. 12), through the orifice of a breast nipple duct. Once properly situated within the mammary duct, the clinician can hold grip 104 against the nipple and pull actuator 105 in the direction away from the breast, partially extending tie rod 106 out of hollow post 102. Retaining clip 116 can then be placed over the extended portion of tie rod 106 to hold the actuator in working position. The transverse dimension of anchor 110 is increased relative to its transverse dimension in resting position, as indicated by the arrows. Expanded anchor 110 holds the device within the mammary duct until the retaining clip 116 is removed, which releases the actuator 105, and bands 111 can flex back into resting positions, reducing the transverse dimension of anchor 110 sufficiently to allow the clinician remove device 100 from the duct.

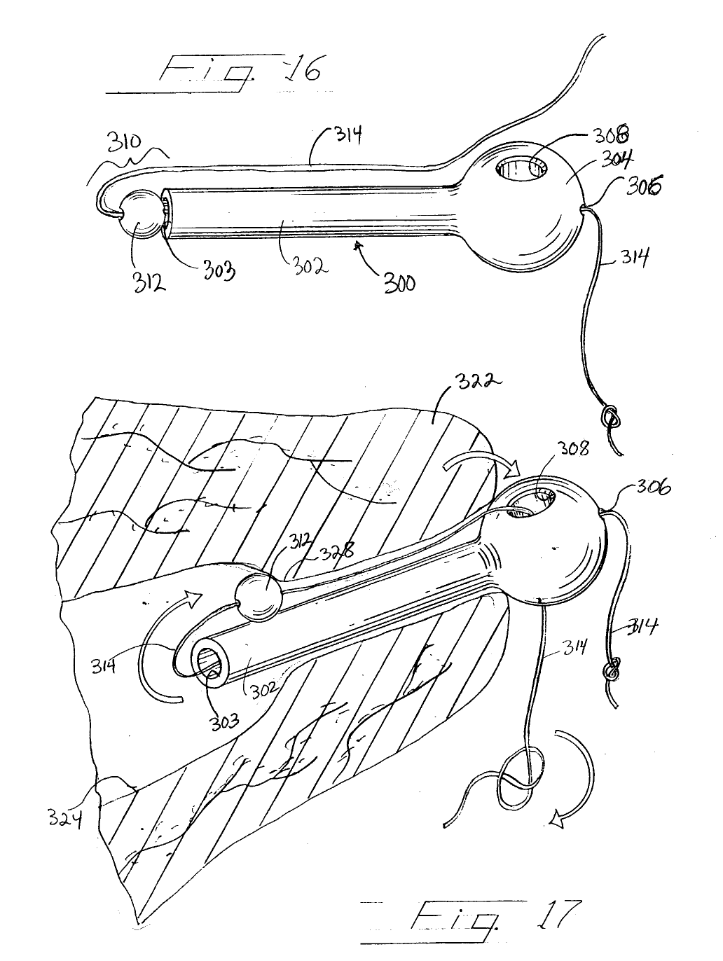

FIGS. 14 and 15 are side views of another preferred embodiment of a device of the present invention having an articulating anchor. Device 200 (shown in resting position in FIG. 14) includes a hollow post 202 defining a passageway 203 and having a hollow grip 204 at its proximal end. Grip 204 is provided with an actuator 205 including a tie rod 206 extending through passageway 203, and terminating at its proximal end in knob 208. Grip 204 is also provided with a cradle 207. Articulating anchor 210 includes distal segment 209 of tie rod 206, which extends out of passageway 203. A plurality of flexible bands 211 are helically arranged around distal segment 209. The proximal end of each band 211 is connected to the distal end 212 of post 202, and the distal end of each band 211 is attached to the distal end 214 of tie rod 206. Knob 207 defines a transverse aperture 215.

FIG. 15 shows device 200 in working position, with actuator 205 displaced proximally, as indicated by the strait arrow, and knob 207 held in cradle 207 to lock actuator 205 in working position.

In use, a clinician can insert device 200, in resting position (FIG. 14), through the orifice of a breast nipple duct. Once properly situated within the mammary duct, the clinician can hold grip 204 against the nipple and pull actuator 205 in the direction away from the breast, partially extending tie rod 206 out of passageway 203. Tie rod 206 is flexible enough to allow knob 205 to be lifted into cradle 207 as indicated by the curved arrow. In working position, bands 211 of anchor 210 are flexed outwardly, increasing the transverse dimension of anchor 210 relative to its transverse dimension in resting position. Expanded, cage-like anchor 210 holds the device within the mammary duct until knob 208 of actuator 205 is released from cradle 207. Upon release of knob 208 from cradle 207, tie rod 206 slides distally within passageway 203, releasing tension from bands 211, which can then flex back into resting position, reducing the transverse dimension of the anchor 210 sufficiently to allow the clinician remove device 200 from the duct.

FIGS. 16 is a side view of another preferred embodiment of the device of the present invention, shown in resting position. Device 300 includes a hollow post 302 defining a passageway 303. Hollow grip 304 at the proximal end of passageway 303 defines an axial through hole 306 and a transverse aperture 308, both of which are in open communication with passageway 303. Anchor bead 312 is held by string 314 in axial alignment with post 302 at the distal end of passageway 303.

FIG. 17 shows device 300 of FIG. 16 in working position within a mammary duct 324 of a human breast 322. Bead anchor 312 is displaced from the longitudinal axis of the post 302, and abutting post 302 at sphincter 328 of duct 324.

The breast nipple duct protection device of the present invention can alternatively include an inflatable anchor. In this embodiment the device includes a hollow post terminating at its proximal end in a hollow grip and collectively defining a passageway. The distal end of the passageway can include an inflatable bladder or balloon in open communication with the passageway. The grip can include an air-tight, removable cap adapted to hold the anchor in an inflated state for a prolonged period of time. The cap can also be a membrane pierceable with a cannula, so that the anchor can be inflated and/or deflated using a syringe.

The foregoing description is to be taken as illustrative, but not limiting. Still other variants within the spirit and scope of the present invention will readily present themselves to those skilled in the art.

I claim:

1. A breast nipple duct protective device which comprises
a post having a longitudinal axis, a distal end and a proximal end; an anchor at the distal end; and a grip at the proximal end; the post having an outside diameter of no more than about 0.5 millimeters; and the anchor having a transverse dimension at least about 1.25 times the outside diameter of the post.

2. The breast nipple duct protective device in accordance with claim 1 wherein the grip has a relatively larger transverse dimension than the outside diameter of the post.

3. The breast nipple duct protective device in accordance with claim 1 wherein the anchor is bulbous.

4. The breast nipple duct protective device in accordance with claim 3 wherein the bulbous anchor is spheroid.

5. The breast nipple duct protective device in accordance with claim 3 wherein the bulbous anchor is ellipsoid.

6. The breast nipple duct protective device in accordance with claim 3 wherein the bulbous anchor is pyriform.

7. The breast nipple duct protective device in accordance with claim 1 wherein the anchor is arciform.

8. The breast nipple duct protective device in accordance with claim 1 wherein the anchor has a blunt, rounded extremity.

9. The breast nipple duct protective device in accordance with claim 1 wherein the post is no more than about 20 millimeters long.

10. The breast nipple duct protective device in accordance with claim 1 wherein the anchor has an adjustable transverse dimension.

11. The breast nipple duct protective device in accordance with claim 1 wherein the device is made of gold.

12. The breast nipple duct protective device in accordance with claim 1 wherein the device is made of titanium.

13. The breast nipple duct protective device in accordance with claim 1 wherein the device is made of stainless steel.

14. The breast nipple duct protective device in accordance with claim 1 wherein the device is made of platinum.

15. The breast nipple duct protective device in accordance with claim 1 wherein the device is made of a physiologically tolerable plastic material.

16. The breast nipple duct protective device in accordance with claim 1 wherein the grip is provided with a transverse aperture.

17. The breast nipple duct protective device in accordance with claim 1 wherein the post is hollow.

18. The breast nipple duct protective device in accordance with claim 1 wherein the post is pliable.

19. The breast nipple duct protective device in accordance with claim 1 wherein a plastic or metal guide is detachably affixed to the anchor, such that the guide can be left in place in the nipple duct when the device is removed from the breast nipple.

20. The breast nipple duct protective device in accordance with claim 1 wherein the post is about 5 to about 20 millimeters long.

21. The breast nipple duct protective device in accordance with claim 1 wherein the post is about 5 to about 10 millimeters long.

22. The breast nipple duct protective device in accordance with claim 1 wherein the grip, post and anchor collectively define a passageway that is open at both ends.

23. The breast nipple duct protective device in accordance with claim 1 wherein the post and grip are hollow and together define a passageway that is open at both ends; the anchor being a bead held against the open distal end of the passageway and axially aligned with the post when in a resting position, the bead being displaced from the longitudinal axis of the post but abutting the post when in a working position.

24. The breast nipple duct protective device in accordance with claim 23 wherein the bead is held against the open distal end of the passageway by a thread passing through the passageway.

25. A breast nipple duct protective device which comprises
a hollow post having a longitudinal axis, a distal end, a proximal end, and defining a lumen therewithin;
an articulating anchor at the distal end of the post having an adjustable transverse dimension;
a grip at the proximal end of the post;
the grip including an actuator adapted for adjusting the transverse dimension of the articulating anchor and operably connected to the articulating anchor through the lumen of the post; and
the post having an outside diameter of no more than about 0.5 millimeters; the articulating anchor having a minimum transverse dimension that is less than 1.25 times the outside diameter of the post and a maximum transverse dimension of at least about 1.25 times the outside diameter of the post.

26. The breast nipple duct protective device in accordance with claim 25 wherein
the grip defines a threaded aperture at the end of the lumen forming a passageway therewith;
the actuator is threadably engagable with the aperture and operably connected to an elongate rod slidably disposed within the passageway; and
the elongate rod is operably connected to the articulating anchor; such that when the actuator is threaded into the aperture the transverse dimension of the articulating anchor is increased.

27. The breast nipple duct protective device in accordance with claim 25 wherein the articulating anchor is in the form of a flexible loop.

28. The breast nipple duct protective device in accordance with claim 25 wherein
the grip includes an aperture at the end of the lumen forming a passageway therewith;
the actuator is a tie rod moveably disposed within the passageway having a proximal end terminating in a knob external to the grip and a distal segment extending beyond the distal end of the lumen and terminating in a blunt atraumatic end;
the articulating anchor comprising a plurality of resilient bands, each band having a proximal end attached to the distal end of the post and a distal end attached to the blunt atraumatic end of the tie rod, each band being at least as long as the distal segment of the post and being capable of flexing outward when a portion of the distal segment of the tie rod is moved into the passageway.

29. The breast nipple duct protective device in accordance with claim 28 wherein the grip includes a removable retaining clip adapted for holding the tie rod in a position in which a portion of the distal segment of the tie rod is within the passageway.

30. The breast nipple duct protective device in accordance with claim 28 wherein the grip includes a cradle adapted for holding the tie rod in a position in which a portion of the distal segment of the tie rod is within the passageway.

31. The breast nipple duct protective device in accordance with claim 28 wherein each band is helically disposed around the distal segment of the tie rod.

32. The breast nipple duct protective device in accordance with claim 28 wherein each band is longitudinally disposed along the distal segment of the tie rod.

33. The breast nipple duct protective device in accordance with claim 25 wherein the grip defines an aperture at the end of the lumen and forming a passageway therewith; the articulating anchor comprises a plurality of jaws, hingedly attached to the distal end of the post; and the actuator comprises a spring-biased plunger within the passageway, operably connected to the jaws; such that when the spring-biased plunger is held in a compressed position the jaws are closed, and when the spring-biased plunger is in a relaxed position the jaws are open.

34. A breast nipple duct protective device which comprises an elongated rod having rounded termini at both ends thereof, the rod having an outside diameter of no more than about 0.5 millimeters and the termini having an outside diameter at least about 1.25 times the outside diameter of the rod.

35. The breast nipple duct protective device in accordance with claim 34 wherein the rounded termini are spheroid.

36. The breast nipple duct protective device in accordance with claim 34 wherein one of the rounded termini has a relatively larger outside diameter.

37. The breast nipple duct protective device in accordance with claim 34 wherein the rounded termini are bulbous.

38. The breast nipple duct protective device in accordance with claim 34 wherein one of said rounded termini is arciform.

39. The breast nipple duct protective device in accordance with claim 34 wherein the rod is no more than about 20 millimeters long.

40. The breast nipple duct protective device in accordance with claim 34 wherein one of the rounded termini has an adjustable outside diameter.

41. The breast nipple duct protective device in accordance with claim 1 in the form of a decorative breast nipple ornament wherein the grip has a decorative form.

42. The breast nipple duct protective device in accordance with claim 41 wherein the decorative form of the grip includes a ring.

43. The breast nipple duct protective device in accordance with claim 41 wherein the decorative form of the grip is a setting for a gem or semiprecious stone ornament.
